(19) United States
(12) Patent Application Publication
(10) Pub. No.: US 2019/0307610 A1
(43) Pub. Date: Oct. 10, 2019

(54) WOUND TREATING SYSTEM AND METHODS OF USING AND ASSEMBLING

(71) Applicants: John Ho, South San Francisco, CA (US); Rachel M. Dorin, South San Francisco, CA (US)

(72) Inventors: John Ho, South San Francisco, CA (US); Rachel M. Dorin, South San Francisco, CA (US)

(73) Assignee: TeraPore Technologies, Inc., South San Francisco, CA (US)

(21) Appl. No.: 16/463,256

(22) PCT Filed: Nov. 21, 2017
(86) PCT No.: PCT/US2017/062681

§ 371 Date: May 22, 2019

Related U.S. Application Data
(60) Provisional application No. 62/426,935, filed on Nov. 28, 2016.

(51) Int. Cl. A61F 13/00

(52) U.S. Cl. CPC A61F 13/00063; A61F 13/00029; A61F 2013/00255; A61F 2013/00221; A61F 13/00038

(57) ABSTRACT

A wound treating article, system and kit, and methods of assembly and treating are provided where nanoporous isoporous membranes are pathogen tuned are combined with resilient, flexible or elastic supports to provide tailored wound treating bandages and/or kits for the medical industry.

### FIELD OF THE INVENTION

Sterile articles that enable air to contact a wound while contemporaneously regulating moisture in the wound and providing pathogen resistance, and optionally as sensors for monitoring and detecting analytes of interest.

### BACKGROUND OF THE INVENTION

In the health care industry, one of the biggest issues facing healthcare providers is their ability to address chronic or severe wounds, which typically can take months to heal. Currently, the state of the art bandage is a hydrocolloid technology, which is a cellulose-based adhesive that acts as a synthetic scab. The problem with hydrocolloid bandages is that they are made with an occlusive polyurethane backing, which causes maceration (dissolution of tissue due to excessive moisture around the wound site). Healthcare providers therefore need to frequently change bandages to prevent maceration, however frequent dressing changes increases the risk of infection and can disturb the body's natural wound healing process.

Another approach to treating severe wounds is the use of multiple layers of gauze and medical tape. The gauze promotes breathability, but they quickly get saturated with fluids over time and are not resistant to pathogens on the nanoscale. So, healthcare providers must regularly change gauze dressings as well (typically every 8 hours).

There remains a need to provide a tailored, sterile bandage that facilitates air transport to the wound, and at the same time, regulates moisture in the wound to avoid or eliminate maceration (water dissolving tissue around the wound site), for less frequent bandage changes and accelerated healing. There also remains a need to provide broad spectrum bacteria and pathogen resistance without the use of antibacterial coatings that can encourage the growth of antibiotic-resistant strains.

### BRIEF DESCRIPTION OF THE DRAWINGS

FIG. 1 illustrates the multilayer composite bandage of the invention;

FIG. 2 is scanning electron microscope (SEM) image of a nanoporous block copolymer membrane showing highly uniform pore size distribution of the invention.

FIG. 3 is illustrates a further embodiment of the present invention including two distinct membranes with tunable pore size.

### DETAILED DESCRIPTION OF THE INVENTION

Embodiments relate to articles that include at least one isoporous, self-assembled nanoporous membrane and a support, where the membrane and/or support is biocompatible. The tailored articles include external bandages, artificial skin grafts, internal bandages for hernias, and cell scaffolding.

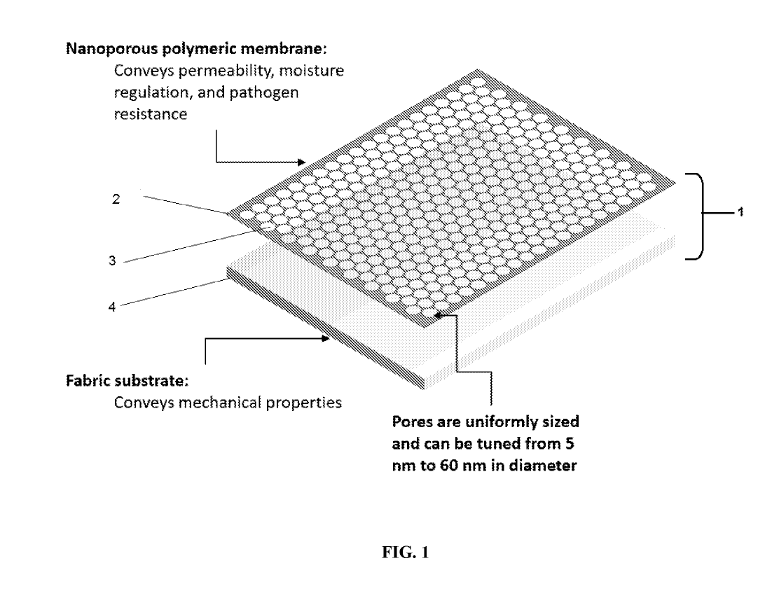

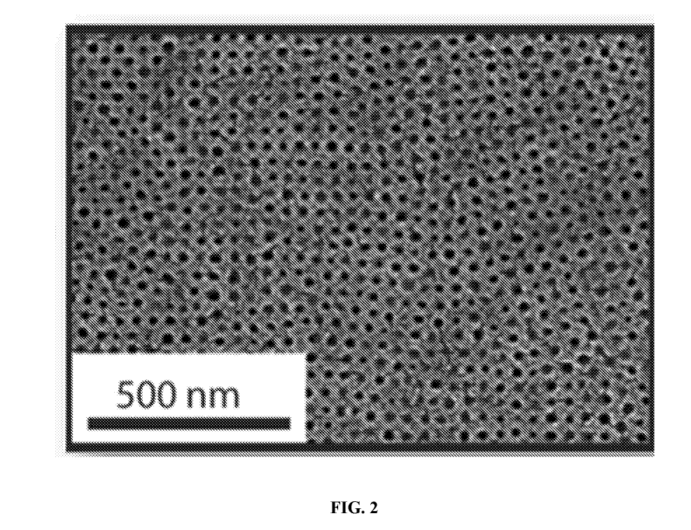

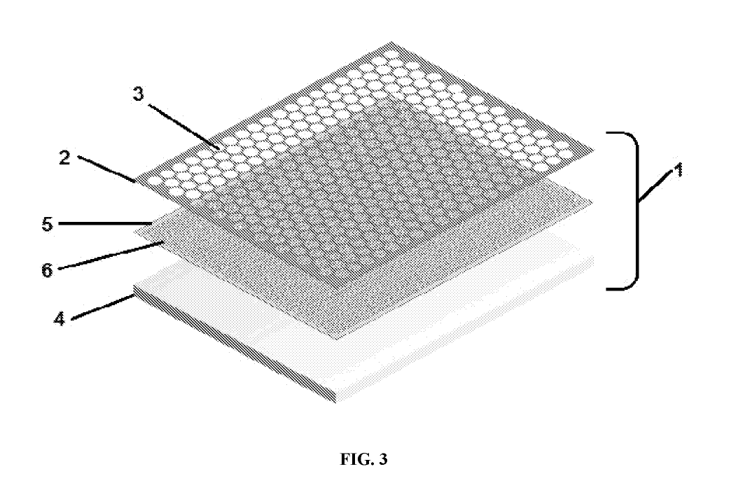

As shown in FIGS. 1 and 3, the article 1 (composite or bandage) includes a membrane 2, tuned pores 3, and support 4. FIG. 3 shows the addition of a second membrane 5, with tuned pores 6 different in size than pores 3. FIG. 2 is an SEM image of the surface of the membrane 2 surface.

More specifically the present inventions provide bandages and kits including isoporous, self-assembled nanoporous membrane having tuned pore sizes ranging from 1 to 60, 100, 200 nm and at least one layer of a flexible, resilient, and/or elastic support layer, which can also be biocompatible. At least one of the support and membrane layers can include an agent that minimizes/reduces sticking to the wound, and can be provided by coating, spraying, or directly incorporating the agent with at least one of the isoporous membrane and support layers. In the context of the invention, isoporous means having a substantially narrow pore diameter distribution.

The membranes of the present invention can be biocompatible for contact with skin/wound or internal use, can be formed from polymeric components that render them biodegradable for temporary use (i.e. synthetic scab), can include antibacterial/microbial agents for sterilizing a wound, can include non-stick agents for release from wound site, can include stiction agents or coating for adhesion to external or internal tissue, can include agents and provide a pore size suitable for enhancing cell growth, and can include drug eluting small molecule(s) and/or biologic(s).

The additional functionality of the membrane of the present invention can include biocompatibility, antibacterial/microbial activity, wound release, drug elution, cell growth enhancement and can be provided by thin film coatings (e.g., dip coating, spray coating, vapor deposition), surface modification (e.g., covalent modification, grafting both to and from, electrostatic attraction (e.g. layer-by-layer, small molecule electrostatic adhesion), varying the membrane polymer chemistry, gas cluster ion beam surface modification, and pore size tunability.

When more than one self-assembled nanoporous membrane are combined, each membrane layer can each have a pore size ranging from 1 to 60 nm, or discrete layers can have a specific pore or different pore size ranges (e.g., 1-30 nm, 10-40 nm, 20-50 nm, 20-40 nm, 20-60 nm). Layers of different size pore ranges/sizes can be combined for additional functionality.

In the present invention, a composite bandage system can have a fabric with multiple layers, each layer providing specific properties. At a minimum, the fabric must contain a tailored nanoporous, polymeric membrane (providing breathability ranging from greater than about 960 g/m² to less than 3200 g/m², regulation of moisture, and pathogen resistance), and a porous backing material that provides mechanical/structural properties, e.g., a support. The bandage system can be provided as a continuous roll, scored or unscored. The bandage fabric support can be longitudinally elastic and tearable in warp or weft direction. Alternatively the bandage system can be provided in discrete sizes of any dimension, e.g., 2×2″, 2×4″, 4×4″, 6×8″.

The bandage system of the present invention can also be provided as a kit where pathogen specific bandages can be provided as an assortment of pre-assembled bandages, for specific classes of pathogens, specific pathogen species, and specific pharmaceutical treating agents that can also be tailored for a patient or type of wound. The kit includes different pathogen specific bandages, such as, at least one first bandage where isoporous, self-assembled nanoporous membrane having a pore size of from 1 to 60 nm, at least one second bandage with an isoporous, self-assembled nanoporous membrane with a pore size of from 1 to 100 nm, and additional bandages with an isoporous, self-assembled nanoporous membrane having other pore sizes, including at least a pore size of from 1 to 200 nm. The bandages of the kit can provide at least one of biocompatibility, biodegradability, anti-bacterial/microbial, non-stick, cell growth enhancement and drug elution.

The bandage, whether designed for a broad spectrum of pathogens, or tailored to a specific pathogen would be selected and applied to a wound such that either the tuned nanoporous membrane or support layer would face/contact the wound, in which case the materials used for the facing layer should be biocompatible, non-leaching, and able to come in direct contact with the open wound, shown in FIG. 1. A bio-compatible non-sticking agent, such as a silicone resin, could be combined with either the membrane or support to allow either side of the bandage system to contact a wound.

The nanoporous, polymeric membrane layers should be highly permeable to air, but impervious to pathogens. Pathogens can include bacteria, viruses, protozoa, or algae, generally 0.01 to 0.1 μm in size for most viruses and 0.1 to 20 μm in size for bacteria. Bacteria are spherical (cocci), rod (bacilli) or spiral (spirochetes) in shape, and are also classified as being gram-positive, e.g., Staphylococcus aureus, non-haemolytic streptococci, Beta-haemolytic streptococcus; gram-negative, e.g., Escherichia coli, Pseudomonas aeruginosa, Klebsiella species, Proteus species. Other pathogens includes anerobes, e.g., Bacteroides and Clostridium; and fungi, e.g., Candida albicans and Aspergillus.

Air permeability and pathogen impermeability is achieved through the use of self-assembled block copolymers that form isoporous membranes with pore sizes ranging from 1 to 60 nm, shown in the SEM of FIG. 2. The high density of pores gives the membrane higher permeability while the isoporosity acts as a size selection barrier for various pathogens of interest. The pore sizes can also be uniformly tuned to control moisture transmission above the wound site. The membrane is a multi-block copolymer having at least one hydrogen-bonding block and a hydrophobic block. Suitable hydrogen bonding blocks include, but are not limited to, polyvinylpyridines, polyethylene oxides, polyacrylates and polymethacrylates, as well as lower alkyl substituted polyacrylates and polymethacrylates. Suitable hydrophobic blocks can include, but are not limited to polystyrenes, e.g., polystyrene and poly (alkyl substituted styrene), such as, poly (alpha-methyl styrene); polyethylenes, polypropylenes, polyvinyl chlorides, and polytetrafluoroethylenes including expanded PTFE.

The porous backing material simply acts as a mechanical substrate for the nanoporous, polymeric membrane. It should have pore sizes that are much larger than the polymeric membrane so as not to create a bottleneck for permeability, and it should convey mechanical stability and flexibility to the composite bandage. Suitable materials could include a knitted, woven or nonwoven material, such as gauze, cellulose-based fabrics, cotton, rayon, polyesters, polyethylenes, and open structure polyurethane film, all of which are sufficiently breathable to allow air to easily flow to and access the site of a wound.

Typical bandage sizes could match existing standard sizes (e.g. 4×4″ or 6×8″) or could be envisioned as a wrap with some self-adhesive material. Alternatives include various material compositions for the various layers, thicknesses of the various layers, as well as variations in the pore size range and degree of isoporosity in the membrane layer. Additional layers can also be added to the stack to provide additional functionality (e.g. biocompatibility, medicinal properties, etc.)

Parameters that provide the necessary protection are the use of a nanoporous, polymeric membrane as a bandage material for wound care. The pore size can range from a few nm and up, which will be important to define control of moisture, transmission rates and rejection of various pathogens.

The solvent 1,4-dioxane, alone or combined with tetrahydrofuran (THF), methanol, ethanol, toluene, chloroform, dimethylformamide, acetone, and dimethylsulfoxide, is used as the solvent in preparing an isoporous graded film of multi-block copolymers, and results in thin selective film layer (i.e., a surface layer) having on the order of more than 10¹⁴ nearly monodisperse mesopores/m² above a graded microporous layer.

Hybridization of the isoporous films via homopolymer or small molecule blending enables tuning of pore size, and can result in pure water flux, solute rejection characteristics, and water vapor transport rates (WVTRs). Tuning pore size is accomplished by the incorporation of small molecules, including but not limited to pentadecyl phenol, dodecyl phenol, 2-4′-(hydroxybenzeneazo) benzoic acid (HABA). 1,8-naphthalene-dimethanol, 3-hydroxy-2-naphthoic acid, and 6-hydroxy-2-naphthoic acid; inorganic and organic acids, including but not limited to hydrofluoric acid, hydrochloric acid, nitric acid, formic acid, acetic acid, propionic acid, lower alkyl di-carboxylic acids; bases, including but not limited to pyridine, ammonia, ammonium hydroxide, sodium hydroxide, potassium hydroxide, amines, polyamines (triethylamine, triethanolamine), amides (acetamide, formamide); including, but not limited to glycerol and other polyols, quinones, hydroquinones, catechols, carbohydrate; and small polymers including but not limited to poly acrylic acid, polyvinylpyridine, polyethylene oxide, naturally-derived polymers (cellulose, chitosan, complex carbohydrates)

Another application of the invention is as part of a sensor, e.g. chemical or biochemical detection and/or quantification. For example, activation of a particular response on the material: resistance, capacitance, color, upon interaction of a target species to the material. In this embodiment, the interaction of the target species with the material invokes a detectable change or response of the material (e.g. change in spectrophotometric profile of membrane), allowing the detection and/or quantification of the target species. The target species may be but is not limited to a molecule, biomolecule (e.g. protein), biological structure (e.g. specific cell type), pathogen, chemical structure (e.g. nanoparticle), moiety on a molecule, moiety on a biomolecule, moiety on a biological structure, or moiety on a chemical structure.

In an embodiment, the material is used in a process detecting an analyte of interest contacting a medium containing at least one analyte of interest with the material.

The materials/films of the present invention include integration into textiles, or a sensor device.

In an embodiment, at least one of the membrane or support is coated or impregnated with at least one topical antibiotic. For example, neomycin, polymixin B, mupirocin, bacitracin, erythromycin, or sulfacetamide sodium.

In an embodiment, at least one of the membrane or support is coated or impregnated with at least one clotting agent. For example, thrombin, tannins, metal salts such as zinc and calcium, gelatin, collagen, fibrin, or other blood factors.

In an embodiment, at least one of the membrane or support is coated or impregnated with at least one agent to promote healing. For example, vitamins, proteins, amino acids, enzymes, or drugs. More specifically, some examples include: aloe vera gel or extract, vitamin A, vitamin B1, vitamin B3, glycine, choline salicylate, or collagen.

In an embodiment, at least one of the membrane or support is coated or impregnated with at least one time released drug. For example, compounds for enabling time release or controlled release include: hydroxypropyl methylcellulose, poly(vinyl alcohol), poly(acrylic acid), or waxes.

In an embodiment, at least one of the membrane or support is coated or impregnated with at least one responsively released drug. For example, a drug is released upon exposure to a particular protein or pathogen.

In an embodiment, at least one of the membrane or support is coated or impregnated with at least one antiseptic. For example, benzalkonium chloride, chlorhexidine, alexidine, povidone-iodine, benzethonium chloride, chloroxylenol, alcohols, or triclosan.

In an embodiment, at least one of the membrane or support is coated or impregnated with at least one anesthetic. For example, benzocaine, lidocaine, tetracaine, pramoxine, phenol, menthol, prilocaine, or dyclonine.

In an embodiment, the invention includes an adhesive for affixing the bandage to the body. In some examples, the adhesive is a pressure sensitive adhesive. For example, at least one pressure sensitive adhesive polymer or block copolymers, comprising, for example: poly(acrylates), poly(methacrylates), rubbers, poly(isoprene), poly(butadiene), poly(acrylates), poly(acrylic acid), poly(vinyl acetate), etc.

In an embodiment, the invention is used as a bandage for a burn.

In an embodiment, the invention is used as an oral bandage.

In an embodiment, the invention is used as a bandage for internal body use.

In an embodiment, the bandage is bioabsorbable after a certain period of time. For example, the materials comprise bioabsorbable polymers or copolymers comprising polymers or polymer blocks such as: poly(lactic acid)s, poly(glycolic acid), polyesters, poly(caprolactone), poly(orthoester), poly(hydroxybutyrate valerate), poly(dioxanone), or poly(trimethylene carbonate).

In some embodiments, the bandage has a two-dimensional or three-dimensional geometric arrangement suitable for particular use or body part. For example, a contoured bandage to more appropriately fit a joint.

In an embodiment, at least one of the membrane or support is coated or impregnated with at least one inactive ingredient for storing, diluting, or delivering a coating or impregnation. For example, pure water, glycerol, petroleum jelly, or lanolin.

In an embodiment, at least one of the membrane or support is coated or impregnated with at least one antioxidant for stability and shelf life. For example, ascorbic acid, tocopherols, carotenoids, carotenes, or butylated hydroxytoluene.

In an embodiment, a portion of the bandage has an inlet, valve, or septum such that a substance can be introduced or extracted to or from the bandage or wound without necessitating removing the bandage. In an example, a drug can be injected from a syringe either with or without a needle without removing the bandage. In an example, a sample of bodily fluid can be extracted with a syringe, with or without a needle, from the bandage or wound without removing the bandage.

In an embodiment, the bandage is directly inserted into a wound as a packing.

In an embodiment, the bandage incorporates an indicator. Said indicator, for example, may indicate: a bandage needs changing, lack of moisture, excess moisture, presence of a pathogen, expenditure of drug or topical agent, wound coagulation, etc.

In an embodiment, the bandage is packaged with a hydrating agent or humectant to retain moisture on the membrane. For example, pure water, glycerol, or aloe vera gel.

In an embodiment, the bandage is packaged dry.

What is claimed as new and desired to be protected by Letters Patent of the United States is:

1. A flexible composite bandage, comprising: a) an isoporous, self-assembled nanoporous membrane; b) a flexible and/or elastic support; and c) optionally a biocompatible non-stick agent in at least one of the isoporous membrane and support.

2. The flexible composite bandage of claim 1, comprising: a) an isoporous, self-assembled nanoporous membrane having a pore size of from 1 to 200 nm; b) a flexible and/or elastic support; and c) optionally a biocompatible non-stick agent in at least one of the isoporous membrane and support, wherein at least one of the membrane and the support are biocompatible.

3. The flexible composite bandage of claim 1, comprising: an isoporous, self-assembled nanoporous membrane having a pore size of from 1 to 60 nm; b) a flexible and/or elastic support; and c) optionally a biocompatible non-stick agent in at least one of the isoporous membrane and support, wherein at least one of the membrane and the support are biocompatible.

4. A method of providing a wound treating system, comprising associating a isoporous self-assembled nanoporous membrane having a pore size of from 1 to 200 nm to a flexible and/or elastic support to form a unitary bandage, wherein a biocompatible non-stick agent is associated with at least one of the isoporous membrane and support and at least one of the membrane and support are biocompatible.

5. The bandage according to claim 1, wherein the support comprises at least one layer of an air permeable, self-supporting, biocompatible material.

6. The bandage of claim 1, wherein the isoporous membrane is arranged on an outmost layer.

7. The bandage of claim 5, wherein the support includes a plurality of layers and the isoporous membrane is arranged between two of the plurality of layers.

8. The bandage according to claim 1, wherein a biocompatible non-stick agent is coated on at least one of the isoporous membrane and support.

9. The bandage according to claim 1, wherein the bandage is pathogen specific.

10. The bandage according to claim 1, wherein at least one of the membrane and support include a pharmaceutical agent.

11. The bandage according to claim 1, wherein at least one of the membrane and support include a pharmaceutical drug.

12. A kit comprising different pathogen specific bandages according to claim 1.

13. A method of treating a wound, comprising adhering a bandage according to claim 1 to a wound.

14. A method of treating a wound comprising, selecting a pathogen specific bandage of claim 9, and adhering the pathogen specific bandage to a wound.

15. A kit comprising different pathogen specific bandages, at least one first bandage comprising an isoporous, self-assembled nanoporous membrane having a pore size of from 1 to 60 nm, at least one second bandage comprising an isoporous, self-assembled nanoporous membrane having a pore size of from 1 to 100 nm, an additional bandage comprising an isoporous, self-assembled nanoporous membrane having other pore sizes, including at least a pore size of from 1 to 200 nm.

16. An article, comprising: a) an isoporous, self-assembled nanoporous membrane having a pore size of from 1 to 200 nm; b) a flexible and/or elastic support; wherein the article provides at least one of biocompatibility, biodegradability, anti-bacterial/microbial, non-stick, enhances cell growth, and drug eluting.

17. The kit of claim 16, where the bandage provides at least one of biocompatibility, biodegradability, anti-bacterial/microbial, non-stick, cell growth enhancement and drug elution.

18. A sensor comprising the material of claim 1.

19. A process detecting at least one analyte of interest contacting a medium containing the analyte of interest with the material of claim 1.
